(19) United States
(12) Patent Application Publication
(10) Pub. No.: US 2023/0256228 A1
(43) Pub. Date: Aug. 17, 2023

(54) PERCUTANEOUS CIRCULATORY SUPPORT DEVICE INCLUDING RECONFIGURABLE DISTAL TIP PORTION

(71) Applicant: Boston Scientific Scimed Inc., Maple Grove, MN (US)

(72) Inventors: Brice Lee Shireman, Maple Grove, MN (US); Darrin Dale Beekman, Osseo, MN (US); Matthew Hart, Maple Grove, MN (US)

(21) Appl. No.: 18/109,135

(22) Filed: Feb. 13, 2023

Related U.S. Application Data
(60) Provisional application No. 63/309,719, filed on Feb. 14, 2022.

(51) Int. Cl. A61M 60/148; A61M 60/216

(52) U.S. Cl. CPC A61M 60/148; A61M 60/216

(57) ABSTRACT

A percutaneous circulatory support device includes a housing and an impeller disposed within the housing and being rotatable relative to the housing to cause blood to flow through the housing. A cannula is coupled to the housing, and a distal tip portion is coupled to the cannula opposite the housing. A control wire is movable to modify at least one of a radius, a stiffness, and a shape of the distal tip portion.

### CROSS-REFERENCE TO RELATED APPLICATIONS

This application claims priority to Provisional Application No. 63/309,719, filed Feb. 14, 2022, which is herein incorporated by reference in its entirety.

### TECHNICAL FIELD

The present disclosure relates to percutaneous circulatory support devices. More specifically, the present disclosure relates to percutaneous circulatory support devices including flexible distal tips.

### BACKGROUND

Percutaneous circulatory support devices such as blood pumps can provide transient support for up to approximately several weeks in patients with compromised heart function or cardiac output. Several issues may complicate delivery and operation of blood pumps within the heart, including difficulty with guidewire advancement, trauma to cardiac tissue, oscillation and/or migration of the blood pump resulting in decreased performance of the blood pump, and difficulty with device withdrawal.

### SUMMARY

In an Example 1, a percutaneous circulatory support device comprises: a housing; an impeller disposed within the housing and being rotatable relative to the housing to cause blood to flow through the housing; a cannula coupled to the housing; a distal tip portion coupled to the cannula opposite the housing; and a control wire being movable to modify at least one of a radius, a stiffness, and a shape of the distal tip portion.

In an Example 2, the percutaneous circulatory support device of Example 1, wherein the control wire is a push wire.

In an Example 3, the percutaneous circulatory support device of Example 1, wherein the control wire is a pull wire.

In an Example 4, the percutaneous circulator support device of any of Examples 1-3, wherein the control wire is a first control wire, and further comprising a second control wire being movable to modify a length of the distal tip portion.

In an Example 5, the percutaneous circulatory support device of any of Examples 1-4, wherein the distal tip portion comprises a spiral shape.

In an Example 6, the percutaneous circulatory support device of any of Examples 1-5, wherein the control wire comprises a radiopaque material.

In an Example 7, the percutaneous circulatory support device of any of Examples 1-6, wherein the control wire extends through the cannula.

In an Example 8, the percutaneous circulatory support device of any of Examples 1-7, wherein the control wire extends through the housing.

In an Example 9, a percutaneous circulatory support device comprises: a housing; an impeller disposed within the housing and being rotatable relative to the housing to cause blood to flow through the housing; a cannula coupled to the housing; a distal tip portion coupled to the cannula opposite the housing; a first active control being manipulable to modify at least one of a radius, a stiffness, and a shape of the distal tip portion; and a second active control being manipulable to modify a length of the distal tip portion.

In an Example 10, the percutaneous circulatory support device of Example 9, wherein the distal tip portion comprises a spiral shape.

In an Example 11, the percutaneous circulatory support device of any of Examples 9-10, wherein the second active control is a pull wire.

In an Example 12, the percutaneous circulatory support device of any of Examples 9-11, wherein the pull wire comprises a radiopaque material.

In an Example 13, the percutaneous circulatory support device of any of Examples 9-12, wherein the first active control extends through the cannula.

In an Example 14, the percutaneous circulatory support device of any of Examples 9-13, wherein the first active control extends through the housing.

In an Example 15, the percutaneous circulatory support device of any of Examples 9-14, wherein the second active control extends through the cannula.

In an Example 16, a percutaneous circulatory support device comprises: a housing; an impeller disposed within the housing and being rotatable relative to the housing to cause blood to flow through the housing; a cannula coupled to the housing; a distal tip portion coupled to the cannula opposite the housing; and a control wire coupled to the distal tip portion, the control wire being movable to modify at least one of a radius, a stiffness, and a shape of the distal tip portion.

In an Example 17, the percutaneous circulatory support device of Example 16, wherein the control wire is a push wire.

In an Example 18, the percutaneous circulatory support device of Example 16, wherein the control wire is a pull wire.

In an Example 19, the percutaneous circulator support device of Example 16, wherein the control wire is a first control wire, and further comprising a second control wire being movable to modify a length of the distal tip portion.

In an Example 20, the percutaneous circulatory support device of Example 16, wherein the distal tip portion comprises a spiral shape.

In an Example 21, the percutaneous circulatory support device of Example 16, wherein the control wire comprises a radiopaque material.

In an Example 22, the percutaneous circulatory support device of Example 16, wherein the control wire extends through the cannula.

In an Example 23, the percutaneous circulatory support device of Example 16, wherein the control wire extends through the housing.

In an Example 24, a percutaneous circulatory support devices comprises: a housing; an impeller disposed within the housing and being rotatable relative to the housing to cause blood to flow through the housing; a cannula coupled to the housing; a distal tip portion coupled to the cannula opposite the housing; a first active control coupled to the distal tip portion, the first active control being manipulable to modify at least one of a radius, a stiffness, and a shape of the distal tip portion; and a second active control coupled to the distal tip portion, the second active control being manipulable to modify a length of the distal tip portion.

In an Example 25, the percutaneous circulatory support device of Example 24, wherein the distal tip portion comprises a spiral shape.

In an Example 26, the percutaneous circulatory support device of Example 24, wherein the second active control is a pull wire.

In an Example 27, the percutaneous circulatory support device of Example 26, wherein the pull wire comprises a radiopaque material.

In an Example 28, the percutaneous circulatory support device of Example 24, wherein the first active control extends through the cannula.

In an Example 29, the percutaneous circulatory support device of Example 24, wherein the first active control extends through the housing.

In an Example 30, the percutaneous circulatory support device of Example 24, wherein the second active control extends through the cannula.

In an Example 31, the percutaneous circulatory support device of Example 24, wherein the second active control extends through the housing.

In an Example 32, a method for positioning a percutaneous circulatory support device within a patient comprises: inserting the percutaneous circulatory support device into the vasculature of the patient, the percutaneous circulatory support device comprising a housing, an impeller disposed within the housing and being rotatable relative to the housing to cause blood to flow through the housing, a cannula coupled to the housing, a distal tip portion coupled to the cannula opposite the housing, and a control wire coupled to the distal tip portion; thereafter moving the control wire to modify at least one of a radius, a stiffness, and a shape of the distal tip portion; and thereafter advancing the percutaneous circulatory support device in the vasculature of the patient.

In an Example 33, the method of Example 32, wherein advancing the percutaneous circulatory support device in the vasculature of the patient comprises crossing a heart valve with the distal tip portion.

In an Example 34, the method of Example 33, wherein the heart valve is an aortic valve.

In an Example 35, the method of Example 33, wherein moving the control wire to modify at least one of the radius, the stiffness, and the shape of the distal tip portion comprises positioning the distal tip portion in a prolapsed configuration, and wherein crossing the heart valve with the distal tip portion comprises crossing the heart valve in the prolapsed configuration.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a partial side view of an illustrative percutaneous circulatory support device (also referred to herein, interchangeably, as a “blood pump”) positioned in the aorta and the heart of a patient, in accordance with embodiments of the subject matter disclosed herein.

FIG. 2 is another partial side view of the percutaneous circulatory support device of FIG. 1.

FIG. 3 is a partial side sectional view of the percutaneous circulatory support device of FIG. 1.

FIG. 4 is a partial side view of the percutaneous circulatory support device of FIG. 1 in a first configuration.

FIG. 5 is a partial side view of the percutaneous circulatory support device of FIG. 1 in a second configuration.

FIG. 6 is a partial side view of the percutaneous circulatory support device of FIG. 1 in a third configuration.

FIG. 7 is another partial side sectional view of the percutaneous circulatory support device of FIG. 1.

FIG. 8 is a partial perspective view of another illustrative percutaneous circulatory support device, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

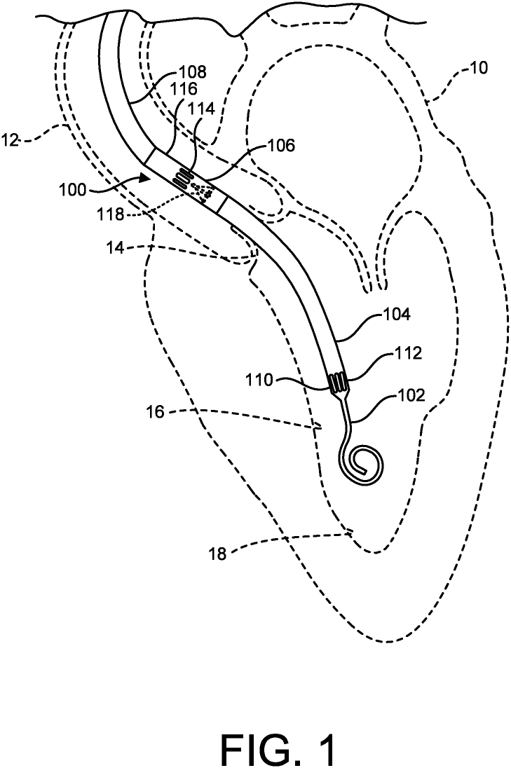

FIG. 1 depicts a portion of an illustrative percutaneous mechanical circulatory support device 100 (also referred to herein, interchangeably, as a “blood pump”), and its relative position in a human heart 10, in accordance with embodiments of the subject matter disclosed herein. The blood pump 100 may be delivered percutaneously by passing through the aorta 12, and positioned within the heart 10 with respect to the aortic valve 14 and the left ventricle 16, as shown in FIG. 1. In some embodiments and as described in further detail below, the blood pump 100 may provide enhanced trackability and may be delivered without using an ancillary guidewire (not shown - that is, a guidewire separate from the blood pump 100). Alternatively, the blood pump 100 may be delivered using an ancillary guidewire.

With continued reference to FIG. 1, the blood pump 100 generally includes a flexible distal tip portion 102 (which may also be referred to as a “guidewire tip”), a cannula 104, an impeller portion 106, and a catheter 108. The cannula 104 may have a flexible construction to facilitate delivery of the blood pump 100. The cannula 104 includes one or more blood inlets 110 located on a distal portion 112 thereof, and one or more blood outlets 114 are located on a housing 116 of the impeller portion 106. The housing 116 carries an impeller 118, and the impeller 118 rotates relative to the housing 116 to cause blood to flow into the inlets 110, through the housing 116, and out of the outlets 114. During operation and as shown in FIG. 1, the blood pump 100 is positioned within the heart 10 such that the inlets 110 are positioned in the left ventricle 16 and the outlets 114 are positioned in the aorta 12. As a result, rotation of the impeller 118 relative to the housing 116 causes blood to flow from the left ventricle 16, through the cannula 104 and the impeller portion 106, and into the aorta 12. The flexible distal tip portion 102 is described in further detail below. However, during operation, the blood pump 100 may be positioned such that the distal tip portion 102 is located in close proximity of, or in contact with, the wall of the left ventricle 16, for example, in the location of the apex 18 of the left ventricle 16.

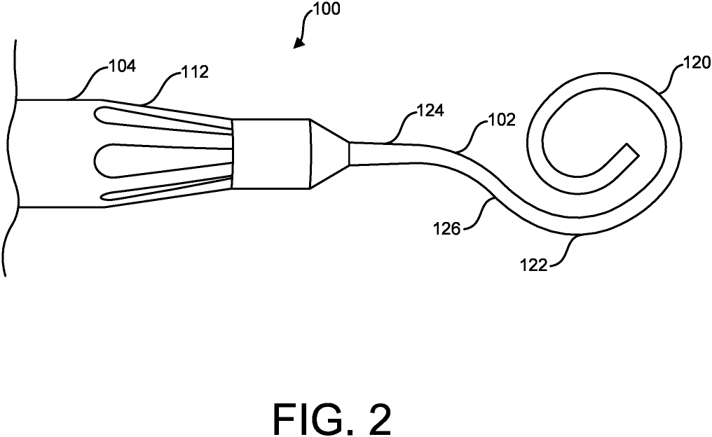

FIG. 2 depicts a side view of a portion of the blood pump 100, specifically the distal portion 112 of the cannula 104 and the distal tip portion 102. Generally, the distal tip portion 102 has a spiral shape. In some embodiments and as illustrated, the distal tip portion 102 includes a distal section 120, an intermediate section 122, and a proximal section 124. The proximal section 124 has a relatively straight shape, the intermediate section 122 has a curved shape, and the distal section 120 has a curved shape that overlaps with the intermediate section 122 but not the proximal section 124. In some embodiments and as illustrated, a bend segment 126 may extend and form a gradually curved transition between the proximal section 124 and the intermediate section 122. In some embodiments, the distal tip portion 102 may be heat set to a specific shape.

The distal tip portion 102 may be made of one or more materials, such as one or more polymers (for example, polyether block amide, thermoplastic polyurethane, or the like), rubbers, plastics, or other materials known to those skilled in the art that allow the distal tip portion 102 to be flexible, relatively soft, and/or collapsible. In some embodiments, the distal tip portion 102 may be constructed of a flexible metal material (for example, Nitinol, Elgiloy, or the like). The distal tip portion 102 may also include features, such as slots or other structures to improve flexibility, collapsibility, performance, durability, or other desirable characteristics. In some embodiments, the distal tip portion 102 may be slotted and made of Nitinol that may be heat set to various shapes.

The proximal section 124 of the distal tip portion 102 is constructed to have moderate stiffness compared to the intermediate section 122 and/or the distal section 120, so that it may act as a dampener for absorbing forces acting on the distal tip portion 102 and the blood pump 100. Moderate stiffness for the proximal section 124 is also beneficial so that the distal tip portion 102 may provide axial strength, which is advantageous for positioning and supporting the cannula 104 in the left ventricle. The stiffness of proximal section 124 may be achieved by constructing the proximal section 124 of one or more materials of appropriate stiffness, by the inclusion of structures, such as reinforcement structures or slots, within the proximal section 124, by combining materials and structures, or by using other techniques known to those of ordinary skill in the art. In some embodiments, the bend segment 126 may also facilitate dampening and absorbing axial forces acting on the distal tip portion 102, and the bend segment 126 may be constructed of similar materials as the proximal section 124.

The intermediate section 122 of the distal tip portion 102 may be constructed to have a stiffness less than the stiffness of the proximal section 124. Such a stiffness for the intermediate section 122 is beneficial so that the intermediate section 122 does not cause trauma or damage when contacting tissue yet provides adequate structural strength for positioning and supporting of the cannula 104 in the left ventricle while also being capable of absorbing forces acting on the distal tip portion 102. The appropriate stiffness of the intermediate section 122 may be achieved by constructing the intermediate section 122 of one or more materials of appropriate stiffness, by the inclusion of structures, such as reinforcement structures or slots, within the intermediate section 122, by combining materials and structures, or by using other techniques known to those of ordinary skill in the art. In general, the intermediate section 122 may be constructed of materials that have a stiffness less than the stiffness of the materials forming the proximal section 124, as measured, for example, by a durometer.

The distal section 120 of the distal tip portion 102 may be constructed to have a stiffness less than the stiffness of the intermediate section 122. Such a stiffness for the distal section 120 is beneficial so that distal section 120 does not cause trauma or damage when contacting tissue yet provides adequate structural strength for positioning and supporting of the cannula 104 in the left ventricle while also being capable of absorbing forces acting on the distal tip portion 102. The appropriate stiffness of the distal section 120 may be achieved by constructing the distal section 120 of one or more materials of appropriate stiffness, by the inclusion of structures, such as reinforcement structures or slots, within the distal section 120, by combining materials and structures, or by using other techniques known to those of ordinary skill in the art. In general, the distal section 120 may be constructed of materials that have a stiffness less than the stiffness of the materials forming intermediate section 122, as measured, for example, by a durometer. In some embodiments, based on the materials used for the reinforcement structures, the inclusion of the structures may aid in the visualization for the distal tip portion 102 under fluoroscopy.

In some embodiments, each section 124, 126, 122, and 120 may have the same stiffness. But as described herein, the stiffness of the proximal section 124 may be greater than the stiffness of the distal section 120. In some embodiments, the stiffness transition may occur in discrete steps along the length of the distal tip portion 102. In some embodiments, the stiffness transition may be gradual or continuous along the length of the distal tip portion 102. In other embodiments, the stiffness transition may be a combination of discrete steps and continuous segments. The decrease in stiffness from the proximal section 124 to the distal section 120 of the distal tip portion 102 may be achieved through a variety of ways, such as decreasing wall thickness of the distal tip portion 102 from the proximal section 124 to the distal section 120, decreasing stiffness of material along the length of distal tip portion 102 without using discrete segments (for example, via intermittent layer coextrusion), changing a pattern or construction of the walls of the distal tip portion 102 along the length of distal tip portion 102, changing a pattern or construction of internal support structures within the walls of the distal tip portion 102 along the length of the distal tip portion 102, heat treating the distal tip portion 102 or sections thereof to change the material properties of the distal tip portion 102 or sections thereof, or by any other method known to those of ordinary skill in the art.

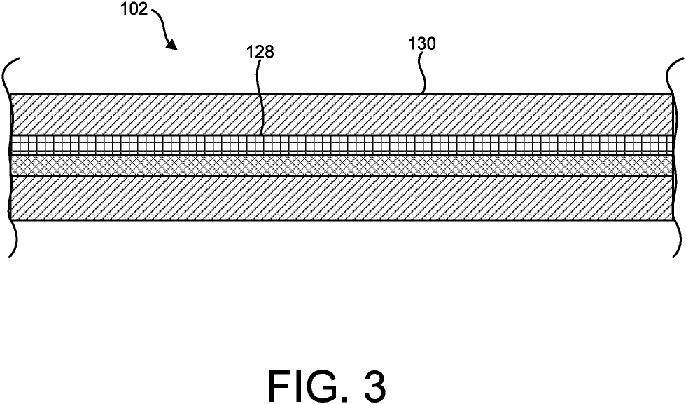

The distal tip portion 102 includes one or more active controls for reconfiguring the distal tip portion 102. More specifically, the active controls manipulable to modify one or more of the radius, the stiffness, the shape, and the length of the distal tip portion 102 (that is, to partially or fully retract the distal tip portion 102 into the cannula 104). Referring to FIG. 3, the active controls may include one or more control wires 128, 130, such as push wires or pull wires, or telescoping tubes. In some embodiments, a first control wire 128 may be a push wire that is manipulable to modify one or more of the radius, the stiffness, and the shape of the distal tip portion 102, and a second control wire 130 may be a pull wire that is manipulable to modify the length of the distal tip portion 102. In some embodiments, the control wires 128, 130 are constructed of a radiopaque material.

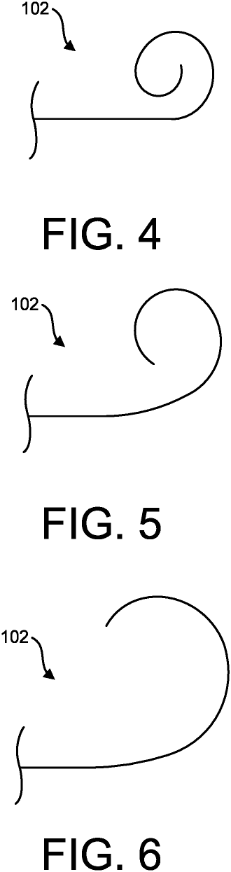

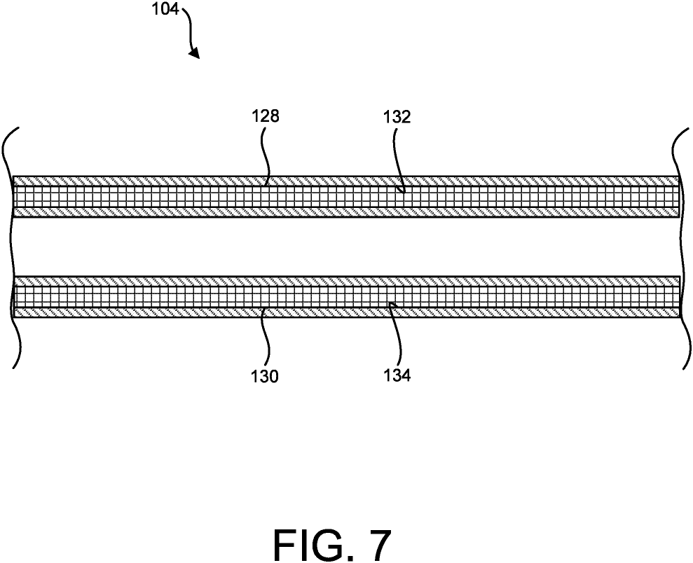

FIGS. 4-6 depict various exemplary configurations of the distal tip portion 102. FIG. 4 depicts a first configuration in which the distal tip portion 102 form a relatively small spiral shape. In the first configuration, the active controls, such as the control wires 128, 130 (shown elsewhere), may be absent from the distal tip portion 102. FIG. 5 depicts a second configuration in which the distal tip portion 102 forms a larger spiral shape. In the second configuration, one or more of the active controls, such as the control wires 128, 130, may be inserted or partially inserted in the distal tip portion 102. FIG. 6 depicts a third configuration in which the distal tip portion 102 forms an even larger spiral shape. In the third configuration, one or more additional active controls, such as the control wires 128, 130, may be inserted in the distal tip portion 102.

FIG. 7 depicts a side sectional view of a portion of the cannula 104. In some embodiments and as illustrated, the active controls, such as the control wires 128, 130, extend through the cannula 104. More specifically, the first control wire 128 and the second control wire 130 may extend through a first channel 132 and a second channel 134, respectively, formed in a coating of the cannula 104. In some embodiments, the active controls, such as the control wires 128, 130, similarly extend through the housing 116 of the impeller portion 106 (shown elsewhere).

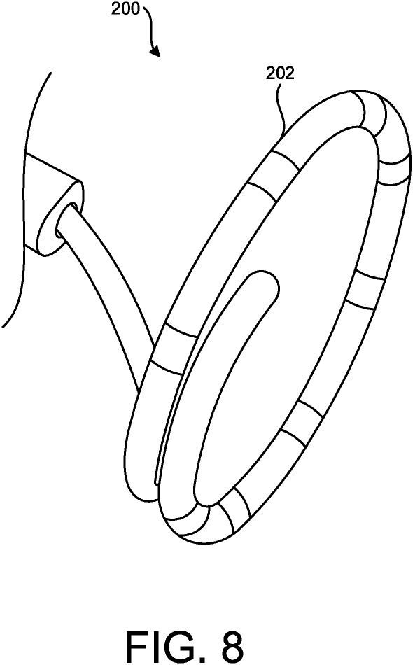

FIG. 8 depicts a perspective view of a distal tip portion 202 of a blood pump 200, in accordance with embodiments of the subject matter disclosed herein. The blood pump 200 may be the same or similar to the blood pump 100, except that the distal tip portion 202 has a different shape than the distal tip portion 102. More specifically, the distal tip portion 202 has a lasso-like shape. In some embodiments, the distal tip portion 202 couples to one or more active controls, such as push wires, pull wires, or telescoping tubes (not shown), for reconfiguring the distal tip portion 202. More specifically, the active controls are manipulable to modify one or more of the radius, the stiffness, the shape, and the length of the distal tip portion 202.

In some embodiments, blood pumps in accordance with embodiments of the subject matter disclosed herein may lack active controls and instead include passive controls for reconfiguring the distal tip portions. In these embodiments, the distal tip portions assume the shape of, or a shape similar to, the passive controls. Such passive controls include, for example, a guidewire (not shown) that is received in a lumen of a distal tip portion. Introduction of the guidewire into the distal tip portion may reconfigure, more specifically expand or straighten, the distal tip portion.

The distal tip portions described herein may provide one or more of various advantages. For example, the distal tip portions may be reconfigured to inhibit the distal tip portions from causing trauma or damage to vascular or cardiac tissues and structures during delivery or operation of the blood pumps, and may assist in delivery and withdrawal of the blood pumps from the vascular and cardiac structures. As another example, flexible, relatively soft, and/or reconfigurable distal tip portions also helps maintain blood pumps in proper position within the heart by absorbing or dampening forces, such as blood flow or ventricular wall contact, acting on the blood pumps when the distal tip portions are positioned against a wall of the left ventricle. If the cannula is constructed to be flexible, there may be movement or oscillation of the blood pump within the heart due to blood flow and contraction of the heart. The flexible, relatively soft, and/or reconfigurable distal tip portions also account for and minimize resistance to lateral contraction forces acting on the tips during contraction of the left ventricle. The distal tip portions may significantly reduce such movement or oscillation by supporting the distal tip portions of the blood pumps against a wall of the left ventricle, thereby stabilizing the blood pumps. Such stabilization may increase efficiency, performance, and/or longevity of the blood pumps. As yet another example, the distal tip portions may facilitate crossing the aortic valve in a prolapsed configuration (that is, with distal ends of the distal tip portions facing away from the aortic valve).

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A percutaneous circulatory support device, comprising:
a housing;
an impeller disposed within the housing and being rotatable relative to the housing to cause blood to flow through the housing;
a cannula coupled to the housing;
a distal tip portion coupled to the cannula opposite the housing; and
a control wire coupled to the distal tip portion, the control wire being movable to modify at least one of a radius, a stiffness, and a shape of the distal tip portion.

2. The percutaneous circulatory support device of claim 1, wherein the control wire is a push wire.

3. The percutaneous circulatory support device of claim 1, wherein the control wire is a pull wire.

4. The percutaneous circulator support device of claim 1, wherein the control wire is a first control wire, and further comprising a second control wire being movable to modify a length of the distal tip portion.

5. The percutaneous circulatory support device of claim 1, wherein the distal tip portion comprises a spiral shape.

6. The percutaneous circulatory support device of claim 1, wherein the control wire comprises a radiopaque material.

7. The percutaneous circulatory support device of claim 1, wherein the control wire extends through the cannula.

8. The percutaneous circulatory support device of claim 1, wherein the control wire extends through the housing.

9. A percutaneous circulatory support device, comprising:
a housing;
an impeller disposed within the housing and being rotatable relative to the housing to cause blood to flow through the housing;
a cannula coupled to the housing;
a distal tip portion coupled to the cannula opposite the housing;
a first active control coupled to the distal tip portion, the first active control being manipulable to modify at least one of a radius, a stiffness, and a shape of the distal tip portion; and
a second active control coupled to the distal tip portion, the second active control being manipulable to modify a length of the distal tip portion.

10. The percutaneous circulatory support device of claim 9, wherein the distal tip portion comprises a spiral shape.

11. The percutaneous circulatory support device of claim 9, wherein the second active control is a pull wire.

12. The percutaneous circulatory support device of claim 11, wherein the pull wire comprises a radiopaque material.

13. The percutaneous circulatory support device of claim 9, wherein the first active control extends through the cannula.

14. The percutaneous circulatory support device of claim 9, wherein the first active control extends through the housing.

15. The percutaneous circulatory support device of claim 9, wherein the second active control extends through the cannula.

16. The percutaneous circulatory support device of claim 9, wherein the second active control extends through the housing.

17. A method for positioning a percutaneous circulatory support device within a patient, comprising:
inserting the percutaneous circulatory support device into the vasculature of the patient, the percutaneous circulatory support device comprising a housing, an impeller disposed within the housing and being rotatable relative to the housing to cause blood to flow through the housing, a cannula coupled to the housing, a distal tip portion coupled to the cannula opposite the housing, and a control wire coupled to the distal tip portion; thereafter
moving the control wire to modify at least one of a radius, a stiffness, and a shape of the distal tip portion; and thereafter
advancing the percutaneous circulatory support device in the vasculature of the patient.

18. The method of claim 17, wherein advancing the percutaneous circulatory support device in the vasculature of the patient comprises crossing a heart valve with the distal tip portion.

19. The method of claim 18, wherein the heart valve is an aortic valve.

20. The method of claim 18, wherein moving the control wire to modify at least one of the radius, the stiffness, and the shape of the distal tip portion comprises positioning the distal tip portion in a prolapsed configuration, and wherein crossing the heart valve with the distal tip portion comprises crossing the heart valve in the prolapsed configuration.
